(19) United States
(12) Patent Application Publication
(10) Pub. No.: US 2022/0175275 A1
(43) Pub. Date: Jun. 9, 2022

(54) LOWER LIMB REHABILITATION SYSTEM BASED ON AUGMENTED REALITY AND BRAIN COMPUTER INTERFACE

(71) Applicants: Kaohsiung Medical University, Kaohsiung (TW); NATIONAL YANG MING CHIAO TUNG UNIVERSITY, Hsinchu City (TW)

(72) Inventors: CHIA-HSIN CHEN, Kaohsiung City (TW); LI-WEI KO, Hsinchu City (TW); YI-JEN CHEN, Kaohsiung City (TW); WEI-CHIAO CHANG, New Taipei City (TW); BO-YU TSAI, Kaohsiung City (TW); KUEN-HAN YU, Chiayi County (TW)

(21) Appl. No.: 17/536,460

(22) Filed: Nov. 29, 2021

(30) Foreign Application Priority Data
Dec. 4, 2020 (TW) 109142906

(51) Int. Cl. A61B 5/11; A61B 5/369; G06T 19/00; G02B 27/01; G06N 20/00; A61B 5/00

(52) U.S. Cl. CPC A61B 5/112; A61B 5/369; G06T 19/006; A61B 2505/09; G06N 20/00; A61B 5/742; A61B 5/1124; G02B 27/017

(57) ABSTRACT

A lower limb rehabilitation system based on augmented reality and a brain computer interface includes a display, a plurality of motion sensors, a brain wave monitor, and an analysis platform. The display is configured to receive and play a virtual scene video to guide a user to perform gait rehabilitation training. The plurality of motion sensors is configured to sense gait data. The brain wave monitor is configured to record an electroencephalogram signal by detecting an electric current change in a brain wave of the user. The analysis platform is configured to compare the gait data with the virtual scene video to determine the accuracy of footsteps of the user and provide feedback. The analysis platform inputs the electroencephalogram signal to a machine learning model to quantify the electroencephalogram signal into an index value representing a lower limb motor function of the user.

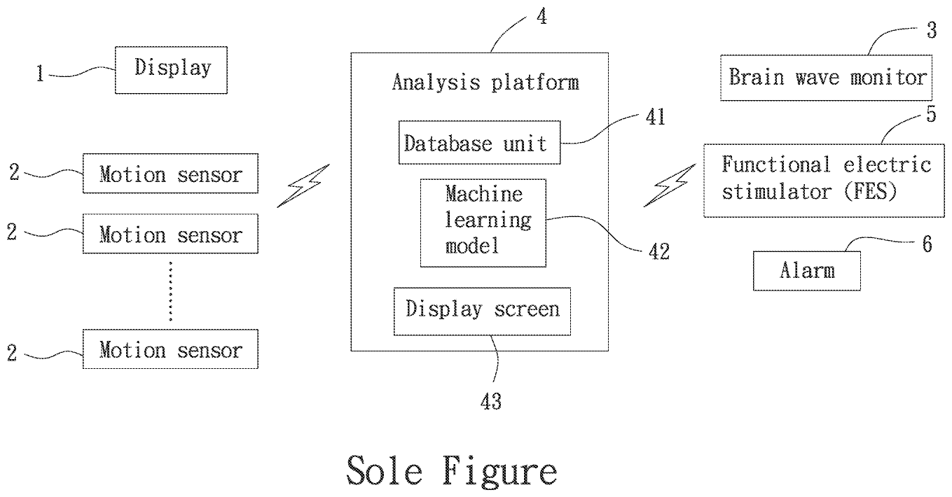

### CROSS REFERENCE TO RELATED APPLICATION

The application claims the benefit of Taiwan application serial No. 109142906, filed on Dec. 4, 2020, and the entire contents of which are incorporated herein by reference.

### BACKGROUND OF THE INVENTION

### 1. Field of the Invention

The present invention relates to a rehabilitation system and, more particularly, to a lower limb rehabilitation system based on augmented reality and a brain computer interface by using an augmented reality technology to assist a patient in rehabilitation.

### 2. Description of the Related Art

Patients having brain trauma, a spinal cord injury, and other osteoarticular diseases have situations of an unsteady gait, erroneous gait postures, walking difficulties, or the like. A conventional rehabilitation method is to perform one-on-one training between a rehabilitation therapist and a patient. The rehabilitation therapist pastes footprint labels on the ground and guides the patient to perform gait training along the footprint labels. Functional magnetic resonance imaging (fMRI) is used for detecting a recovery situation in an injured area of the brain of the patient, to evaluate a recovery status of the lower limb of the patient.

For the above conventional rehabilitation method, the patient needs the assistance of the rehabilitation therapist for rehabilitation, and therefore needs to commute between a home and a hospital frequently. In addition, the fMRI is performed once at an interval of a plurality of months to determine the recovery situations of the brain and the lower limb of the patient, so that the patient cannot learn his/her current recovery status immediately. Therefore, the conventional rehabilitation method has the problems such as a waste of time and costs, an incapability of immediately learning the effectiveness of rehabilitation, and the like.

Thus, it is necessary to provide a lower limb rehabilitation system based on augmented reality and a brain computer interface to resolve the above problems.

### SUMMARY OF THE INVENTION

To solve the above problems, it is an objective of the present invention to provide a lower limb rehabilitation system based on augmented reality and a brain computer interface, to assist a patient in performing rehabilitation by using an augmented reality technology.

It is another objective of the present invention to provide a lower limb rehabilitation system based on augmented reality and a brain computer interface, so that the patient can learn a recovery status of the lower limb by means of spontaneous detection at home.

It is yet another objective of the present invention to provide a lower limb rehabilitation system based on augmented reality and a brain computer interface, so that a tibialis anterior muscle of the patient can be electrically stimulated, to assist the patient in completing gait training.

As used herein, the term “a”, “an” or “one” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

As used herein, the term “database unit” described in the present invention is to collect a set of related electronic data and store the electronic data in a hard disc, a memory, or a combination thereof. Related processing is performed on the electronic data by means of grammatical functions provided by a database management system (DBMS), such as adding, reading, searching, updating, deleting, and the like. The DBMS is capable of managing the electronic data by using different data structures, such as a relational database, a hierarchical database, a network database, or an object-oriented database. The present invention takes the relational DBSMS as an example for description below, but is not limited in this regard.

As used herein, the term “coupling” described in the present invention means that two devices may be connected in any direct or indirect manner to transfer data to each other. For example, a first device is coupled to a second device. In the present invention, it should be understood that the first device may be directly connected to the second device. For example, the first device may be connected to the second device by using a wired entity (such as an electric wire, a flat cable, a trace, and a twisted-pair cable). Alternatively, the first device may be indirectly connected to the second device by using other devices or some connection means. For example, the first device may be connected to the second device by using a wireless medium (such as Wi-Fi and Bluetooth) or a heterogeneous network. One of ordinary skill in the art may perform selection according to a normal connection means by which the devices are to be connected.

A lower limb rehabilitation system based on augmented reality and a brain computer interface in the present invention includes a display, a plurality of motion sensors, a brain wave monitor, and an analysis platform. The display is configured for a user to wear and configured to receive and play a virtual scene video for the user to watch, to guide the user to perform gait rehabilitation training. The plurality of motion sensors is respectively disposed at a plurality of parts of a lower limb of the user and configured to sense gait data. The brain wave monitor is configured to record an electroencephalogram (EEG) signal by detecting an electric current change in a brain wave of the user. The EEG signal is a brain wave signal in a brain motor area of the user. The analysis platform is coupled to the display, the plurality of motion sensors, and the brain wave monitor. The analysis platform is configured to store a plurality of virtual scene videos by using a database unit, and select the virtual scene videos from the database unit and transmit the virtual scene videos to the display; receive the gait data sensed by the plurality of motion sensors and compare the gait data with the virtual scene videos, to determine the accuracy of footsteps of the user according to a virtual sign generated by the virtual scene videos and provide the user with feedback; input the EEG signal to a machine learning model, so that the machine learning model quantifies the EEG signal into an index value, with the index value used for representing a lower limb motor function of the user; and output the index value.

Thus, according to the lower limb rehabilitation system based on augmented reality and a brain computer interface in the present invention, the display can be used to play the virtual scene videos for the user to watch, to guide the user to perform gait rehabilitation training. Gait data sensed by the plurality of motion sensors is compared with the virtual scene videos to determine the accuracy of footsteps of the user according to a virtual sign generated by the virtual scene videos, and provide the user with feedback on the rehabilitation training. The analysis platform detects, by using the brain wave monitor, the EEG signal of the user after performing the gait rehabilitation training, and inputs the EEG signal to the machine learning model to evaluate and quantify the effectiveness of the gait rehabilitation training of the user, thereby obtaining and outputting an index value representing the lower limb motor function of the user. In this way, according to the present invention, the user may directly use the lower limb rehabilitation system based on augmented reality and a brain computer interface at home without the need to go to the hospital. Therefore, the time and costs for commuting between the home and the hospital can be saved, and the effectiveness of the gait rehabilitation training of the user can be learned immediately.

In an example, the analysis platform has a display screen. The display screen is configured to visualize an index value result determined by the machine learning model, for a rehabilitation therapist to observe a brain electrophysiological activity during the training of the user. Thus, by means of data visualization, the rehabilitation therapist can identify the effectiveness of rehabilitation of the user more visually.

In an example, the plurality of virtual scene videos has different rehabilitation difficulty levels, and the analysis platform is configured to select the virtual scene video having the corresponding difficulty level according to the index value of the user, for the user to perform gait rehabilitation training in conformity with a current status of the user. Thus, the rehabilitation therapist may select the virtual scene video having a proper rehabilitation difficulty level according to the rehabilitation recovery status of the user, for the user to perform the gait rehabilitation training. In this way, the user can be prevented from secondary damage as a result of an increased training difficulty level, and a poor training effect as a result of a decreased training difficulty level can be avoided.

In an example, each virtual scene video has a music rhythm, and the analysis platform is configured to control the display to synchronously play the virtual scene video and the music rhythm, so that the user performs the gait rehabilitation training with beats of the music rhythm. Thus, the user can feel interested and challenged, so that the willing of the user for rehabilitation is stimulated. Therefore, the rehabilitation efficiency is enhanced.

In an example, the plurality of motion sensors is respectively disposed on a waist, two thighs, two calves, and at least one instep of the user, and a plurality of reference planes is defined by positions of the plurality of motion sensors. Thus, gait data of the parts of the user such as bilateral hip joints, knee joints, and an ankle joint on the affected side can be measured, to determine the accuracy of footsteps of the user more accurately. Therefore, the accuracy of estimating the effectiveness of rehabilitation is improved.

In an example, the display is configured to project and superimpose, onto the real world, a plurality of virtual signs in the virtual scene video, for the user to walk along the plurality of virtual signs. Thus, the user can perform the gait rehabilitation training at home.

In an example, the lower limb rehabilitation system based on augmented reality and a brain computer interface according to the present invention further includes a functional electric stimulator coupled to the analysis platform. The functional electric stimulator is disposed on the lower limb of the user, and is configured to electrically stimulate a tibialis anterior muscle of the user, to cause the tibialis anterior muscle of the user to contract. Thus, the system can avoid foot drop when the user performs the gait rehabilitation training and can assist the user in walking.

In an example, the lower limb rehabilitation system based on augmented reality and a brain computer interface according to the present invention further includes an alarm coupled to the analysis platform. The analysis platform is configured to evaluate whether the index value is greater than an index threshold, and if an evaluation result is “No”, the analysis platform controls the alarm to transmit a warning signal to remind a rehabilitation therapist to adjust a parameter of the functional electric stimulator. Thus, the system can improve the effectiveness of rehabilitation of the user.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

The sole FIGURE is a block diagram of a system according to a preferred embodiment of the present invention.

In the various FIGURES of the drawings, the same numerals designate the same or similar parts. Furthermore, when the terms “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the invention.

### DETAILED DESCRIPTION OF THE INVENTION

Referring to the FIGURE, a preferred embodiment of a lower limb rehabilitation system based on augmented reality and a brain computer interface according to the present invention includes a display 1, a plurality of motion sensors 2, a brain wave monitor 3, and an analysis platform 4. The display 1, the plurality of motion sensors 2, and the brain wave monitor 3 are coupled to the analysis platform 4.

The display 1 is provided for a user to wear, and is configured to receive and play a virtual scene video for the user to watch, to guide the user to perform gait rehabilitation training. In this embodiment, the display 1 may project and superimpose, onto the real world, a plurality of virtual signs in the virtual scene video, for the user to walk along the plurality of virtual signs. For example, the display 1 may be smart glasses such as Microsoft HoloLens, and have functions such as augmented reality (AR), gesture recognition, voice recognition, iris recognition, and the like. The display 1 may also be other head-up or head-mounted displays having the same functions. The present invention is not limited in this regard.

The plurality of motion sensors 2 is respectively disposed at a plurality of parts of a lower limb of the user and is configured to sense gait data. In this embodiment, each motion sensor 2 may be a six-axis sensor. The six-axis sensor includes a three-axis accelerometer and a three-axis gyroscope, such as MPU6050 launched by InvenSense Inc. Preferably, each motion sensor 2 may be a nine-axis sensor. The nine-axis sensor may be a combination of a three-axis accelerometer, a three-axis gyroscope, and a three-axis magnetometer, a combination of a six-axis accelerometer and a three-axis gyroscope, or a combination of a three-axis accelerometer and a six-axis gyroscope.

Specifically, a quantity of the plurality of motion sensors 2 is preferably six to seven. The motion sensors may be respectively disposed at a waist, two thighs, two calves, and at least one instep of the user. Each two motion sensors 2 may form a pair and define a reference plane to conclude a coordinate coefficient of the knee joint of the user, thereby comprehensively measuring a changing angle at joints of the lower limb. For example, on one lower limb, the motion sensors 2 at the waist and the thigh may form a first pair, the motion sensors 2 at the thigh and the calf can form a second pair, and the motion sensors 2 at the calf and the instep can form a third pair. For instance, two motion sensors 2 are respectively disposed at the thigh and the calf to record a change of position coordinate of the thigh and the calf on the same plane, thereby concluding the coordinate coefficient of the knee joint. Namely, the detection of the changing angle at joints is performed by analyzing the position coordinate of two adjacent parts of the lower limb. The gait data may include information such as a position, an angle, a speed, and an acceleration of the joints of the lower limb of the user when walking. Therefore, data such as a step speed, a step frequency, a step distance, and symmetry of the user can be calculated accordingly.

The brain wave monitor 3 is configured to record an EEG signal by detecting an electric current change in a brain wave of the user. The EEG signal refers to an EEG signal in a brain motor area of the user. In this embodiment, the brain wave monitor 3 may be a wearable brain wave electrode cap, and is configured to record brain wave power values in frequency bands such as α, β, δ and θ in the EEG signal of the user.

The analysis platform 4 is coupled to the display 1, the plurality of motion sensors 2, and the brain wave monitor 3. In this embodiment, a Raspberry Pi 3/4 may be used as the analysis platform 4. The analysis platform 4 stores a plurality of virtual scene videos by using a database unit 41. The analysis platform 4 selects one of the virtual scene videos from the database unit 41, and transmits the virtual scene video to the display 1, so that the display 1 plays the virtual scene video for the user to perform gait rehabilitation training according to the virtual scene video. The analysis platform 4 receives the gait data sensed by the plurality of motion sensors 2 to compare the gait data with the virtual scene video, to determine the accuracy of footsteps of the user according to a virtual sign generated by the virtual scene video and provide the user with feedback. The form of feedback may include a voice prompt or a video prompt, and the present invention is not limited thereto.

The analysis platform 4 inputs the EEG signal to a machine learning model 42, so that the machine learning model 42 quantifies the EEG signal into an index value. The index value is used for representing a lower limb motor function of the user. In this embodiment, a larger index value indicates that the lower limb motor function of the user approximates that of a healthy person. The analysis platform 4 outputs the index value. The machine learning model 42 is, for example, but not limited to being trained by using a support vector machine (SVM). One of ordinary skill in the art may understand the technology of the SVM, and details will not be described herein.

It is to be noted that the plurality of virtual scene videos may have different rehabilitation difficulty levels such as elementary, intermediate, advanced and a customized rehabilitation difficulty level. The analysis platform 4 may select the virtual scene videos having the corresponding difficulty level according to the index value of the user, for the user to perform gait rehabilitation training in conformity with a current status of the user. On the other hand, each virtual scene video may have a music rhythm. The display 1 synchronously plays the music rhythm while playing the virtual scene video, so that the user can perform the gait rehabilitation training with beats of the music rhythm.

The analysis platform 4 of the lower limb rehabilitation system based on augmented reality and a brain computer interface according to the present invention may further include a display screen 43. The display screen 43 is configured to visualize an index value result determined by the machine learning model 42, for a rehabilitation therapist to observe a brain electrophysiological activity during the training of the user. The display screen 43 may be, for example, but is not limited to a common computer screen, or mobile devices having a display function, such as a smart phone, a tablet, or a laptop. The user may capture a picture displayed on the display screen 43 and transmit the picture to the rehabilitation therapist, for the rehabilitation therapist to observe the brain electrophysiological activity of the user.

The lower limb rehabilitation system based on augmented reality and a brain computer interface according to the present invention may further include a functional electric stimulator (FES) 5. The FES 5 is disposed on the lower limb of the user and is coupled to the analysis platform 4. The analysis platform 4 may control the FES 5 to electrically stimulate a tibialis anterior muscle of the user, to cause the tibialis anterior muscle of the user to contract. In this way, the system can avoid foot drop when the user performs the gait rehabilitation training, and can assist the user in walking. Specifically, the analysis platform 4 may analyze, according to the gait data such as ankle joint angles and hip joint angles of the user, whether the user has the foot drop. If an analysis result is “Yes”, the FES 5 is controlled to electrically stimulate the tibialis anterior muscle of the user. If the analysis result is “No”, no extra operation is performed.

The lower limb rehabilitation system based on augmented reality and a brain computer interface according to the present invention may further include an alarm 6 coupled to the analysis platform 4. The analysis platform 4 can evaluate whether the index value is greater than an index threshold. If an evaluation result is “Yes”, the analysis platform 4 does not need to perform an extra operation. If the evaluation result is “No”, the analysis platform 4 may control the alarm 6 to transmit a warning signal to remind the rehabilitation therapist to adjust a parameter of the FES 5, thereby ensuring that the user can finish the gait rehabilitation training as scheduled. The alarm 6 may be, for example, but is not limited to a light-emitting diode, a buzzer, or a combination thereof, and is configured to transmit a warning signal such as warning light, a warning sound, or a combination thereof. The present invention is not limited in this regard.

In use of the lower limb rehabilitation system based on augmented reality and a brain computer interface according to the present invention, the user (for example, a stroke patient) wears the display 1 and the brain wave monitor 3 on the head, and the plurality of motion sensors 2 is disposed at body parts such as a waist, a thigh, a calf, an instep, and the like. The user or the rehabilitation therapist controls the analysis platform 4 to select a virtual scene video in conformity with the current rehabilitation difficulty level of the user, so that the analysis platform 4 transmits the virtual scene video to the display 1. The virtual scene video may be constructed by Unity. The display 1 projects and superimposes, onto the real world, two virtual channels and a plurality of virtual signs in the virtual scene video. The plurality of virtual signs is respectively located in one of the virtual channels, and move toward the user along the virtual channels with the music rhythms. In this way, the user can perform gait rehabilitation training with the beats of the music rhythms according to the plurality of virtual signs. When the user performs gait rehabilitation training, the analysis platform 4 receives the gait data sensed by the plurality of motion sensors 2, and analyzes whether an angle of bending the knee joint of the user reaches a predetermined threshold (for example, 30 degrees), and the virtual sign does not move to the rear of the user yet. If the analysis result is “Yes”, the analysis platform 4 controls the virtual scene video to generate a virtual object, and causes the virtual object and the virtual sign on the corresponding virtual channel to offset each other, thereby obtaining a rehabilitation score. If the analysis result is “No”, no extra operation is performed.

The analysis platform 4 inputs the EEG signal sensed by the brain wave monitor 3 to the machine learning model 42, so that the machine learning model 42 quantifies the EEG signal into an index value (for example, in a range of 1 to 100) used for representing a lower limb motor function. In this way, the user can learn the rehabilitation level of the lower limb. Further, during the gait rehabilitation training, the user may attach the FES 5 to the tibialis anterior muscle to facilitate contraction of the tibialis anterior muscle through electrical stimulation, to avoid the foot drop. Further, the analysis platform 4 may evaluate whether the index value is greater than an index threshold (for example, 70). If the evaluation result is “No”, the alarm 6 is controlled to transmit a warning signal, to remind the user and the rehabilitation therapist to adjust a parameter of the FES 5, thereby improving the effectiveness of rehabilitation of the user.

Based on the above, according to the lower limb rehabilitation system based on augmented reality and a brain computer interface of the present invention, the display can be used to play the virtual scene videos for the user to watch, to guide the user to perform gait rehabilitation training. Gait data sensed by the plurality of motion sensors is compared with the virtual scene videos to determine the accuracy of footsteps of the user according to the virtual sign generated by the virtual scene videos, and provide the user with the feedback on the rehabilitation training. The analysis platform detects, by using the brain wave monitor, the EEG signal of the user after performing the gait rehabilitation training, and inputs the EEG signal to the machine learning model to evaluate and quantify the effectiveness of the gait rehabilitation training of the user, thereby obtaining and outputting the index value representing the lower limb motor function of the user. In this way, according to the present invention, the user may directly use the lower limb rehabilitation system based on augmented reality and a brain computer interface at home without the need to go to the hospital. Therefore, the time and costs for commuting between the home and the hospital can be saved, and the effectiveness of the gait rehabilitation training of the user can be learned immediately.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. A lower limb rehabilitation system based on augmented reality and a brain computer interface, comprising:
a display for a user to wear and configured to receive and play a virtual scene video for the user to watch, to guide the user to perform gait rehabilitation training;
a plurality of motion sensors respectively disposed at a plurality of parts of a lower limb of the user and configured to sense gait data;
a brain wave monitor configured to record an electroencephalogram signal by detecting an electric current change in a brain wave of the user, wherein the electroencephalogram signal is a brain wave signal in a brain motor area of the user; and
an analysis platform coupled to the display, the plurality of motion sensors, and the brain wave monitor, wherein the analysis platform is configured to: store a plurality of virtual scene videos by using a database unit, and select the virtual scene videos from the database unit and transmit the virtual scene videos to the display; receive the gait data sensed by the plurality of motion sensors and compare the gait data with the virtual scene videos, to determine the accuracy of footsteps of the user according to a virtual sign generated by the virtual scene videos and provide the user with feedback; input the electroencephalogram signal to a machine learning model, so that the machine learning model quantifies the electroencephalogram signal into an index value, wherein the index value is used for representing a lower limb motor function of the user; and output the index value.

2. The lower limb rehabilitation system based on augmented reality and the brain computer interface as claimed in claim 1, wherein the analysis platform has a display screen, and the display screen is configured to visualize an index value result determined by the machine learning model, for a rehabilitation therapist to observe a brain electrophysiological activity during the training of the user.

3. The lower limb rehabilitation system based on augmented reality and the brain computer interface as claimed in claim 1, wherein the plurality of virtual scene videos has different rehabilitation difficulty levels, and the analysis platform is configured to select the virtual scene video having the corresponding difficulty level according to the index value of the user, for the user to perform gait rehabilitation training in conformity with a current status of the user.

4. The lower limb rehabilitation system based on augmented reality and the brain computer interface as claimed in claim 1, wherein each virtual scene video has a music rhythm, and the analysis platform is configured to control the display to synchronously play the virtual scene video and the music rhythm, so that the user performs the gait rehabilitation training with beats of the music rhythm.

5. The lower limb rehabilitation system based on augmented reality and the brain computer interface as claimed in claim 1, wherein the plurality of motion sensors is respectively disposed on a waist, two thighs, two calves, and at least one instep of the user, and a plurality of reference planes is defined by positions of the plurality of motion sensors.

6. The lower limb rehabilitation system based on augmented reality and the brain computer interface as claimed in claim 1, wherein the display is configured to project and superimpose, onto the real world, a plurality of virtual signs in the virtual scene video, for the user to walk along the plurality of virtual signs.

7. The lower limb rehabilitation system based on augmented reality and the brain computer interface as claimed in claim 1, the lower limb rehabilitation system further comprising a functional electrical stimulator coupled to the analysis platform, wherein the functional electrical stimulator is disposed on the lower limb of the user, and is configured to electrically stimulate a tibialis anterior muscle of the user, to cause the tibialis anterior muscle of the user to contract.

8. The lower limb rehabilitation system based on augmented reality and the brain computer interface as claimed in claim 7, the lower limb rehabilitation system further comprising an alarm coupled to the analysis platform, wherein the analysis platform is configured to evaluate whether the index value is greater than an index threshold, and if an evaluation result is no, the analysis platform controls the alarm to transmit a warning signal to remind a rehabilitation therapist to adjust a parameter of the functional electric stimulator.
